(19) United States
(12) Patent Application Publication
(10) Pub. No.: US 2023/0375571 A1
(43) Pub. Date: Nov. 23, 2023

(54) GDF3 AS BIOMARKER AND BIOTARGET IN POST-ISCHEMIC CARDIAC REMODELING

(71) Applicants: INSERM (INSTITUT NATIONAL DE LA SANTÉ ET DE LA RECHERCHE MÉDICALE), Paris (FR); ASSISTANCE PUBLIQUE-HÔPITAUX DE PARIS (APHP), Paris (FR); UNIVERSITÉ PARIS CITÉ, Paris (FR)

(72) Inventor: Jean-Sébastien HULOT, PARIS (FR)

(21) Appl. No.: 18/247,945

(22) PCT Filed: Oct. 4, 2021
(86) PCT No.: PCT/EP2021/077267

§ 371 Date: Apr. 5, 2023

(30) Foreign Application Priority Data
Oct. 5, 2020 (EP) 20306156.9

(51) Int. Cl. G01N 33/68; C07K 16/22

(52) U.S. Cl. CPC G01N 33/6893; C07K 16/22; G01N 2800/32; G01N 2800/56; G01N 2333/475

(57) ABSTRACT

Markers of an intense scarring process in the early phase post-myocardial infarction (MI) are still undetermined, and the identification of patients at higher risk of developing large adverse fibrotic remodeling and heart failure remains challenging. Here, the inventors demonstrate the modulation in the paracrine behavior of resident PW1+ cells in scarring cardiac tissue post-MI and the differential abundance of 12 candidate markers in their secretome. Of these, growth differentiation factor 3 (GDF3), a member of transforming growth factor-β family, upregulates proliferation of cardiac fibroblasts, which are instrumental in fibrosis. GDF3 is upregulated in the scarred tissue and plasma of mice and humans post-MI, with the highest plasma levels predicting higher fibrotic cardiac remodeling and cardiac dilation. The inventors thus reveal the previously unidentified function of GDF3 in predicting adverse fibrotic cardiac remodeling post-MF Thus the present invention relates to the use of GDF3 as biomarker and biotarget in post-ischemic cardiac remodeling.

### FIELD OF THE INVENTION

The present invention is in the field of medicine, in particular cardiology.

### BACKGROUND OF THE INVENTION

Acute myocardial infarction (MI) is characterized with death of billion cardiomyocytes that activates an adaptive reparative process, or cardiac fibrosis, ultimately leading to the replacement of the dead myocardium with a collagen-based scar¹. A major goal of modern cardiovascular research is to regress myocardial scar, which is an important clinical predictor of mortality and sudden cardiac death². While markers indicative of necrosis (increase in cardiac troponin)³ and cardiac dysfunction (increase in brain natriuretic peptide)⁴ have been identified, markers of an intense scarring process in the early phase post-MI are still undetermined, and the identification of patients at higher risk of developing large adverse fibrotic remodeling and heart failure remains challenging.

Growth differentiation factor 3 (GDF3), a member of TGF-β superfamily, also referred to as Vgr-2, was initially identified to modulate early embryonic development, adipose-tissue homeostasis and energy balance by interacting with cell membrane activin receptor-like kinase type I receptor B (ACVR1B, ALK4) and ACVR1C (ALK7) (Andersson, O.; Korach-Andre, M.; Reissmann, E.; Ibanez, C. F.; Bertolino, P. Growth differenti-ation factor 3 signals through ALK7 and regulates accumulation of adipose tissue and diet-induced obesity. Proc. Natl. Acad. Sci. USA 2008, 105, 7252-7256). Recently it has been shown that acute administration of rGDF3 into endotoxic shock mice could increase survival outcome and improve cardiac function through anti-inflammatory response by suppression of M1 macrophage phenotype (Wang L, Li Y, Wang X, et al. GDF3 Protects Mice against Sepsis-Induced Cardiac Dysfunction and Mortality by Suppression of Macrophage Pro-Inflammatory Phenotype. Cells. 2020; 9(1):120. Published 2020 Jan. 3). However the role of GDF3 in post-ischemic cardiac remodeling has never been investigated.

### SUMMARY OF THE INVENTION

The present invention is defined by the claims. In particular the present invention relates to the use of GDF3 as biomarker and biotarget in post-ischemic cardiac remodeling.

### DETAILED DESCRIPTION OF THE INVENTION

Regression of myocardial scar after acute myocardial infarction (MI) is a major cardiovascular research goal, but the incomplete understanding of diverse sources of cardiac fibrosis poses a huge challenge. This alone has restricted identification of patient subgroup at a higher risk of developing adverse fibrotic remodeling and heart failure. Here, the inventors demonstrate the modulation in the paracrine behavior of resident PW1⁺ cells in scarring cardiac tissue post-MI and the differential abundance of 12 candidate markers in their secretome. Of these, growth differentiation factor 3 (GDF3), a member of transforming growth factor-β family, upregulates proliferation of cardiac fibroblasts, which are instrumental in fibrosis. GDF3 is upregulated in the scarred tissue and plasma of mice and humans post-MI, with the highest plasma levels predicting higher fibrotic cardiac remodeling and cardiac dilation. The inventors thus reveal the previously unidentified function of GDF3 in predicting adverse fibrotic cardiac remodeling post-MI.

### Methods of Diagnosis

The first object of the present invention relates to a method of determining whether a patient who experienced a myocardial infarction has or is at risk of having adverse post-ischemic cardiac remodeling comprising determining the level of GDF3 in a sample obtained from the patient wherein said level indicates whether the subject has or is at risk of having adverse post ischemic cardiac remodeling.

As used herein, the term “subject”, “individual” or “patient” is used interchangeably and refers to any subject for whom diagnosis, treatment, or therapy is desired, particularly humans. Other subjects may include cattle, dogs, cats, guinea pigs, rabbits, rats, mice, horses, and the like. In some preferred embodiments, the subject is a human.

As used herein, the term “myocardial infarction” has its general meaning in the art and relates to the irreversible necrosis of the myocardium as a result of prolonged ischemia due to coronary thrombosis, i.e. the development of a clot in a major blood vessel serving the heart.

As used herein, the term “adverse post-ischemic cardiac remodeling” has its general meaning in the art and refers to the prominent changes that occur after myocardial infarction and that could be deleterious for the cardiac function. Cardiac remodeling involves molecular, cellular, and interstitial changes that manifest clinically as changes in size, shape, and function of the heart which occur after myocardial infarction. For instance, ventricular remodeling involves progressive enlargement of the ventricle with depression of ventricular function. Myocyte function in the myocardium remote from the initial myocardial infarction becomes depressed. In particular, adverse post-ischemic cardiac remodeling includes arrhythmias, cardiac dilation (assessed by left ventricular end diastolic volume indexed on body surface area or LVEDVi) and cardiac dysfunction (left ventricular ejection fraction or EF). Typically, adverse post-ischemic cardiac remodeling is defined as a >20% increase in left ventricular end-diastolic volume (LVEDV) at 6 months as compared to the initial evaluation (see EXAMPLE).

In some embodiments, the method of the present invention is particularly suitable for determining whether the patient is at risk of having heart failure after myocardial infarction.

As used herein, the term “heart failure” or “HF” has its general meaning in the art and embraces congestive heart failure and/or chronic heart failure. Functional classification of heart failure is generally done by the New York Heart Association Functional Classification (Criteria Committee, New York Heart Association. Diseases of the heart and blood vessels. Nomenclature and criteria for diagnosis, 6th ed. Boston: Little, Brown and co, 1964; 114). This classification stages the severity of heart failure into 4 classes (I-IV). The classes (I-IV) are: Class I: no limitation is experienced in any activities; there are no symptoms from ordinary activities; Class II: slight, mild limitation of activity; the patient is comfortable at rest or with mild exertion; Class III: marked limitation of any activity; the patient is comfortable only at rest; Class IV: any physical activity brings on discomfort and symptoms occur at rest.

As used herein, the term “risk” in the context of the present invention, relates to the probability that an event will occur over a specific time period and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion. “Risk evaluation” or “evaluation of risk” in the context of the present invention encompasses making a prediction of the probability, odds, or likelihood that an event or disease state may occur, the rate of occurrence of the event or conversion from one disease state to another. Risk evaluation can also comprise prediction of future clinical parameters, traditional laboratory risk factor values, or other indices of relapse, either in absolute or relative terms in reference to a previously measured population. The methods of the present invention may be used to make continuous or categorical measurements of the risk of conversion, thus diagnosing and defining the risk spectrum of a category of subjects defined as being at risk of conversion. In the categorical scenario, the invention can be used to discriminate between normal and other subject cohorts at higher risk. In some embodiments, the present invention may be used so as to discriminate those at risk from normal.

As used herein, the term “sample” as used herein refer to a biological sample obtained for the purpose of in vitro evaluation. Typical biological samples to be used in the method according to the invention are blood samples (e.g. whole blood sample or serum sample).

As used herein the term “blood sample” means any blood sample derived from the subject. Collections of blood samples can be performed by methods well known to those skilled in the art. In some embodiments, the blood sample is a serum sample or a plasma sample.

In some embodiments, the level of GDF3 is determined 1, 2, 3, 4, 5, 6, 7, 8, 9, 10, 11, 12, 13, 14, or 15 days after the myocardial infarction.

As used herein, the term “GDF3’ has its general meaning in the art and refers to the growth/differentiation factor 3. An exemplary amino acid sequence for GDF3 is shown as SEQ ID NO:1. Typically, the term “GDF3” is to be understood by the presence of the mature domain that ranges from the amino acid residue at position 251 to the amino acid residue at position 364 in SEQ ID NO:1.

>sp|Q9NR23|GDF3_HUMAN Growth/differentiation
factor 3 OS = Homo sapiens OX = 9606 GN = GDF3
PE = 1 SV = 3
SEQ ID NO: 1
MLRFLPDLAFSFLLILALGQAVQFQEYVFL
QFLGLDKAPSPQKFQPVPYILKKIFQDREA
AATTGVSRDLCYVKELGVRGNVLRFLPDQG
FFLYPKKISQASSCLQKLLYFNLSAIKERE
QLTLAQLGLDLGPNSYYNLGPELELALFLV
QEPHVWGQTTPKPGKMFVLRSVPWPQGAVH
FNLLDVAKDWNDNPRKNFGLFLEILVKEDR
DSGVNFQPEDTCARLRCSLHASLLVVTLNP
LTISLNSSNYAFMQALMHAVDPEIPQAVCI
PTKLSPISMLYQDNNDNVILRHYEDMVVDE
CGCG

The level of GDF3 in the sample can be determined using methods known in the art, e.g., using quantitative immunoassay methods such as enzyme linked immunosorbent assays (ELISAs), immunoprecipitations, immunofluorescence, enzyme immunoassay (EIA), radioimmunoassay (RIA), and Western blot analysis.

In some embodiments, the methods include contacting the sample with an agent that selectively binds to the GDF3 protein in particular to its mature domain as defined above (such as an antibody or antigen-binding portion thereof) with a sample, to evaluate the level of protein in the sample. In some embodiments, the antibody bears a detectable label. Antibodies can be polyclonal, or more preferably, monoclonal. An intact antibody, or an antigen-binding fragment thereof (e.g., Fab or F(ab′)2) can be used. As used herein, the term “labeled” with regard to an antibody encompasses direct labeling of the antibody by coupling (i.e., physically linking) a detectable substance to the antibody, as well as indirect labeling of the antibody by reactivity with a detectable substance. Examples of detectable substances are known in the art and include chemiluminescent, fluorescent, radioactive, or colorimetric labels. For example, detectable substances can include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

In some embodiments, high throughput methods, e.g., protein or gene chips as are known in the art (see, e.g., Ch. 12, “Genomics,” in Griffiths et al., Eds. Modern genetic Analysis, 1999, W. H. Freeman and Company; Ekins and Chu, Trends in Biotechnology, 1999; 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of GDF3.

In some embodiments, microfluidic (e.g., “lab-on-a-chip,” “micro-a-fluidic chips”) devices can be used in the present methods for detection and quantification of GDF3 protein in a sample. Such devices have been successfully used for microfluidic flow cytometry, continuous size-based separation, and chromatographic separation. In particular, such devices can be used for the isolation of specific biological particles such as specific proteins (e.g., GDF3) from complex mixtures such as serum (e.g., whole blood, serum, or plasma). A variety of approaches may be used to separate GDF3 proteins from a heterogeneous sample. For example, some techniques can use functionalized materials to capture GDF3 using functionalized surfaces that bind to the target cell population. The functionalized materials can include surface-bound capture moieties such as antibodies or other specific binding molecules, such as aptamers, as are known in the art. Accordingly, such microfluidic chip technology may be used in diagnostic and prognostic devices for use in the methods described herein. For examples, see, e.g., Lion et al., Electrophoresis 24 21 3533-3562 (2003); Fortier et al., Anal. Chem., 77(6):1631-1640 (2005); U.S. Patent Publication No. 2009/0082552; and U.S. Pat. No. 7,611,834. Also included in the present application are microfluidics devices comprising GDF3 binding moieties, e.g., anti-GDF3 antibodies or antigen-binding fragments thereof.

Typically, high levels of GDF3 indicate that the probability that the patient has or is at risk of having adverse post-ischemic cardiac remodeling is high and conversely low levels of GDF3 indicate that the probability that the patient has or is at risk of having adverse post-ischemic cardiac remodeling is low.

As used herein, the term “high” refers to a measure that is greater than normal, greater than a standard such as a predetermined reference value or a subgroup measure or that is relatively greater than another subgroup measure. For example, high GDF3 refers to a measure of GDF3 that is greater than a normal GDF3 measure. A normal GDF3 measure may be determined according to any method available to one skilled in the art. High GDF3 may also refer to a measure that is equal to or greater than a predetermined reference value, such as a predetermined cutoff. High GDF3 may also refer to a measure of GDF3 wherein a high GDF3 subgroup has relatively greater levels of GDF3 than another subgroup. For example, without limitation, according to the present specification, two distinct patient subgroups can be created by dividing samples around a mathematically determined point, such as, without limitation, a median, thus creating a subgroup whose measure is high (i.e., higher than the median) and another subgroup whose measure is low. In some cases, a “high” level may comprise a range of level that is very high and a range of level that is “moderately high” where moderately high is a level that is greater than normal, but less than “very high”.

As used herein, the term “low” refers to a measure that is less than normal, less than a standard such as a predetermined reference value or a subgroup measure that is relatively less than another subgroup measure. For example, low GDF3 means a measure of GDF3 that is less than a normal GDF3 measure in a particular set of samples of patients. A normal GDF3 measure may be determined according to any method available to one skilled in the art. Low GDF3 may also mean a measure that is less than a predetermined reference value, such as a predetermined cutoff. Low GDF3 may also mean a measure wherein a low GDF3 subgroup is relatively lower than another subgroup. For example, without limitation, according to the present specification, two distinct patient subgroups can be created by dividing samples around a mathematically determined point, such as, without limitation, a median, thus creating a group whose measure is low (i.e., less than the median) with respect to another group whose measure is high (i.e., greater than the median).

In some embodiments, the method of the present invention comprises the steps of i) quantifying the level of GDF3 in the sample obtained from the patient ii) comparing the level quantified at step i) with a predetermined reference value and iii) concluding that the patient has or is at risk of having has or is at risk of having adverse post-ischemic cardiac remodeling when the level quantified at step i) is higher than the predetermined reference value or inversely concluding that the patient does not have or is not at risk of having has or is at risk of having adverse post-ischemic cardiac remodeling when the content quantified at step i) is lower than the predetermined reference value.

In some embodiments, the predetermined reference value is a threshold value or a cut-off value that can be determined experimentally, empirically, or theoretically. A threshold value can also be arbitrarily selected based upon the existing experimental and/or clinical conditions, as would be recognized by a person of ordinary skilled in the art. For example, retrospective measurement in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after quantifying the GDF3 level in the sample, one can use algorithmic analysis for the statistic treatment of the GDF3 level determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

The method of the present invention is particularly suitable for identifying patients that may need extra attention and support after myocardial infarction. In particular, the method of the present invention is suitable for determining whether the patient is eligible to a treatment with vasodilators, angiotensin II receptor antagonists, angiotensin converting enzyme inhibitors, aldosterone antagonists, diuretics, hydralazine/nitrates, antithrombolytic agents, β-adrenergic receptor antagonists, α-adrenergic receptor antagonists, calcium channel blockers, etc. Examples of ACE inhibitors include, but are not limited to, captopril, benazepril, enalapril, lisinopril, fosinopril, ramipril, perindopril, quinapril, moexipril, and trandolapril. Examples of ARBs include losartan, candesartan, irbesartan, and valsartan. Examples of beta-blockers suitable include, but are not limited to, alprenolol, carteolol, levobunolol, mepindolol, metipranolol, nadolol, oxprenolol, penbutolol, pindolol, propranolol, sotalol, timolol, acebutolol, atenolol, betaxolol, bisoprolol, esmolol, metoprolol, nebivolol, carvedilol, celiprolol, labetalol, and butaxamine. Examples of diuretics include, but are not limited to, calcium chloride, ammonium chloride, amphotericin B, lithium citrate, Goldenrod, Juniper, dopamine, acetazolamide, dorzolamide, bumetanide, ethacrynic acid, furosemide, torsemide, glucose, mannitol, amiloride, spironolactone, triamterene, bendroflumethiazide, hydrochlorothiazide, caffeine, and theophylline. Examples of anti-arrhythmic drugs include, but are not limited to, disopyramide, procainamide, quinidine, lidocaine, phenytoin, flecainide, propafenone, propranolol, timolol, metoprolol, sotalol, atenolol, amiodarone, sotalol, bretylium, verapamil, and diltiazem. Examples of aldosterone include but are not limited to Spironolactone, Eplerenone, Canrenone and potassium canrenoate as well as Finerenone.

### Methods of Treatment

The second object of the present invention relates to a method of treating adverse post-ischemic cardiac remodeling in a patient who experienced a myocardial infarction comprising administering to the subject a therapeutically effective amount of a GDF3 inhibitor.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

In particular, the method of the present invention is suitable for protecting against or reducing damage to the myocardium after a myocardial infarction, after, during or prior to ischemic reperfusion. More particularly, the method of the present invention is particularly suitable for reducing post ischemic left ventricular remodeling. More particularly, the method of the invention is suitable for increasing the left ventricle ejection fraction (LVEF), and/or for inhibiting left ventricle enlargement, and/or for reducing left ventricle end systolic volume, and/or reducing left ventricle end diastolic volume, and/or for ameliorating left ventricle dysfunction, and/or for improving myocardial contractibility.

As used herein, the term “GDF3 inhibitor” refers to any compound natural or not which is capable of inhibiting the activity or expression of GDF3. The term encompasses any GDF3 inhibitor that is currently known in the art or that will be identified in the future, and includes any chemical entity that, upon administration to a patient, results in inhibition or down-regulation of a biological activity or expression of GDF3.

In some embodiments, the GDF3 inhibitor is an anti-GDF3 neutralizing antibody. In some embodiments, the anti-GDF3 neutralizing antibody binds to the mature domain of GDF3. In some embodiments, the anti-GDF3 neutralizing antibody binds to the amino acid sequence that ranges from the amino acid residue at position 251 to the amino acid residue at position 364 in SEQ ID NO:1.

As used herein, the term “antibody” is thus used to refer to any antibody-like molecule that has an antigen binding region, and this term includes antibody fragments that comprise an antigen binding domain. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments.

As used herein, the term “neutralizing antibody” refers to an antibody that is capable of reducing or inhibiting (blocking) activity or signaling of the ligand as determined by in vivo or in vitro assays.

In some embodiments, the antibody of the present invention is a single domain antibody. As used herein the term “single domain antibody” has its general meaning in the art and refers to the single heavy chain variable domain of antibodies of the type that can be found in Camelid mammals which are naturally devoid of light chains. Such single domain antibodies are also “nanobody®”.

In some embodiments, the antibody of the present invention is a fully human antibody. As used herein, the term “fully human” refers to an immunoglobulin, such as an antibody or antibody fragment, where the whole molecule is of human origin or consists of an amino acid sequence identical to a human form of the antibody or immunoglobulin. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference.

In some embodiments, the antibody of the present invention is a humanized antibody. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference.

In some embodiments, the GDF3 inhibitor is an aptamer. Aptamers are a class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. In this library, each member is a linear oligomer, eventually chemically modified, of a unique sequence. Peptide aptamers consists of a conformationally constrained antibody variable region displayed by a platform protein, such as E. coli Thioredoxin A that are selected from combinatorial libraries by two hybrid methods (Colas et al., 1996).

In some embodiments, the GDF3 inhibitor is an inhibitor of GDF3 expression. An “inhibitor of expression” refers to a natural or synthetic compound that has a biological effect to inhibit the expression of a gene. In a preferred embodiment of the invention, said inhibitor of gene expression is a siRNA, an antisense oligonucleotide or a ribozyme. For example, anti-sense oligonucleotides, including anti-sense RNA molecules and anti-sense DNA molecules, would act to directly block the translation of GDF3 mRNA by binding thereto and thus preventing protein translation or increasing mRNA degradation, thus decreasing the level of GDF3, and thus activity, in a cell. For example, antisense oligonucleotides of at least about 15 bases and complementary to unique regions of the mRNA transcript sequence encoding GDF3 can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. GDF3 gene expression can be reduced by contacting a patient or cell with a small double stranded RNA (dsRNA), or a vector or construct causing the production of a small double stranded RNA, such that GDF3 gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing GDF3. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. In some embodiments, the inhibitor of expression is an endonuclease. The term “endonuclease” refers to enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, and cleave only at very specific nucleotide sequences. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homology-directed repair (HDR). In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences. In some embodiment, the endonuclease is CRISPR-cas9 which is from Streptococcus pyogenes. The CRISPR/Cas9 system has been described in U.S. Pat. No. 8,697,359 B1 and US 2014/0068797. In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

In some embodiments, the GDF3 inhibitor is administered to a subject having one or more signs or symptoms of acute myocardial infarction injury. In some embodiments, the subject has one or more signs or symptoms of myocardial infarction, such as chest pain described as a pressure sensation, fullness, or squeezing in the mid portion of the thorax; radiation of chest pain into the jaw or teeth, shoulder, arm, and/or back; dyspnea or shortness of breath; epigastric discomfort with or without nausea and vomiting; and diaphoresis or sweating.

In some embodiments, the GDF3 inhibitor is administered simultaneously or sequentially (i.e. before or after) with a revascularization procedure performed on the subject. In some embodiments, the subject is administered with the GDF3 inhibitor before, during, and after a revascularization procedure. In some embodiments, the subject is administered with the GDF3 inhibitor as a bolus dose immediately prior to the revascularization procedure. In some embodiments, the subject is administered with the GDF3 inhibitor continuously during and after the revascularization procedure. In some embodiments, the subject is administered with the GDF3 inhibitor for a time period selected from the group consisting of at least 3 hours after a revascularization procedure; at least 5 hours after a revascularization procedure; at least 8 hours after a revascularization procedure; at least 12 hours after a revascularization procedure; at least 24 hours after a revascularization procedure. In some embodiments, the revascularization procedure is selected from the group consisting of percutaneous coronary intervention; balloon angioplasty; insertion of a bypass graft; insertion of a stent; directional coronary atherectomy; treatment with one or more thrombolytic agent(s); and removal of an occlusion.

By a “therapeutically effective amount” is meant a sufficient amount of the active ingredient for treating or reducing the symptoms at reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination with the active ingredients; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about mg/kg of body weight per day, especially from about 0.001 mg/kg to 7 mg/kg of body weight per day.

Typically the active ingredient of the present invention (e.g. GDF3 inhibitor) is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the active ingredients of the invention can be administered in a unit administration form, as a mixture with conventional pharmaceutical supports. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

The invention will be further illustrated by the following figures and examples. However, these examples and figures should not be interpreted in any way as limiting the scope of the present invention.

### FIGURES

FIG. 1. GDF3 is a circulating factor secreted post-MI and may predict adverse cardiac remodeling in humans. a. Representative western blots and quantification of mature GDF3 in non-failing (NF) (n=6) and failing hearts (HF) (n=9) of patients. *P<0.05 as determined by Mann Whitney test. b. GDF3 levels in non-remodelers (n=24) and remodelers (n=56) at day 4 post-MI. *P=0.05 as analyzed with Mann Whitney non-parametric t-test. c. ROC curve for the discrimination between remodelers and non-remodelers. d-g. LVEDVi (d), LVEF (e), infarct size (f), and number of akinetic segments (g) at 6 months post-MI in patients from low GDF3 (<1375 pg/mL) and high (>1375 pg/mL) GDF3 groups. *P<0.05, **P<0.01 by Mann Whitney non-parametric t-test.

### EXAMPLE

### Material & Methods

All procedures and animal care protocols were approved by our institutional research committee (CEEA34 and French ministry of research, N° 2019050221153452) and conformed the animal care guideline in Directive 2010/63/EU European Parliament. All animals received humane care in compliance with the “Principles of Laboratory Animal Care” formulated by the National Society for Medical Research and the “Guide for the Care and Use of Laboratory Animals” prepared by the Institute of Laboratory Animal Resources and published by the National Institutes of Health (NIH Publication No. 86-23, revised 1996).

### Animal Study Design

Left anterior descending artery (LAD) surgery was performed on male 8- or 13-week-old male C57BL/6J and PW1-reporter (PW1^{nLacZ}) mice, which were anesthetized in an induction chamber with 2% isoflurane mixed with 1.0 L/min 100% O₂ and placed on a supine position on a heating pad to maintain body temperature. The mice were intubated with endotracheal tube and then connected to a rodent ventilator (180 breaths/min and a tidal volume of 200 μL). During surgical procedure, anesthesia was maintained at 1.5-2% isoflurane with O₂. The chest was accessed from the left side through the intercostal space, and the pericardium incised. The LAD was exposed and encircled with an 8.0 prolene suture at the proximal position. The suture was briefly snared to confirm the ligation by blanching the arterial region. Mice were analyzed 7 days after LAD permanent ligation. Blood samples were collected in heparin-coated Eppendorf tubes and immediately centrifuged at 200×g for 15 min at 4° C. to separate the plasma, which was stored at −80° C. until analysis. Hearts were excised and immediately digested for FACS sorting or qPCR analysis.

### Cell Isolation and Fluorescence-Activated Cell Sorting (FACS)

PW1⁺CD51⁺ cardiac cell sorting was performed as previously described²⁹. Briefly, small cell suspensions were prepared from total heart upon atria removal from 8-week-old PW1^{nLacZ} mice. The ventricles were enzymatically digested with collagenase II and dissociated. The following antibodies were used for cell sorting: BUV737-tagged anti-CD31 (1:100 dilution; BD Bioscience), BUV395-tagged anti-TER119 (1:50 dilution, BD Biosciences), phycoerythrin-cyanin7-tagged anti-CD45 (1:500 dilution; eBiosciences). To detect β-gal reporter activity, cells were incubated with the fluorescent substrate 5-dodecanoylaminofluorescein di-β-D-galactopyranoside (C₁₂FDG) at 37° C. for 1 h. The different populations were gated, analyzed, and sorted on a FACS Aria II cytometer (BD Biosciences).

### CyQUANT™ Cell Proliferation Assay

FACS-sorted PW1⁺ and PW1⁻(FDG⁻) cells were seeded in 24-well plates at a density of 15,000 cells/well and cultured under normal conditions in Dulbecco's modified Eagle's medium (DMEM) supplemented with 10% fetal bovine serum (FBS, Sigma) and 1% penicillin and streptomycin (Sigma) for 5 days. The medium was collected and used to incubate serum-starved MEFs (cultured for 24 h under normal conditions and then serum starved for 24 h) for 24 h. The proliferation of MEFs was evaluated using the CyQUANT cell proliferation assay as per the manufacturer's instructions. MEFs incubated with complete medium served as the control.

### RNA-Sequencing and Bioinformatics Analysis

In total, 300 ng of total RNA extracted from freshly isolated cells with SureSelect Strand-Specific RNA kit (Agilent) was used to prepare a library, according to the manufacturer's instructions. The resulting library was quality checked and quantified by peak integration on Bioanalyzer High sensitivity DNA labchip (Agilent). A pool of equal quantity of 12 purified libraries was prepared, and each library was tagged with a different index. The mRNA pool libraries were finally sequenced on Illumina Hiseq 1500 instrument using a rapid flowcell. The pool was loaded on two lanes of the flowcell. A paired-end sequencing of 2×100 bp was performed.

After discarding reads that did not pass the Illumina filters and trimming low-quality sequenced bases (q<28) using the Cutadapt program³⁰, we restricted our downstream analyses to reads with lengths greater than 90 bp. Selected reads were mapped to a murine reference transcriptome that was generated by the RSEM package³¹ from the full mouse reference genome and the gtf transcript annotations file from ENSEMBL³². Alignment and estimation of transcripts abundance in each of the 12 processed samples were performed using the RSEM program. Transcripts with abundance counts higher than 10 in more than two samples (N=36,948) were considered as expressed and retained for further analysis. Abundances of transcripts assigned to the same gene were combined together, leading to the profiling of 16,403 genes. Analyses were conducted under the R environment (version 3.2.2).

Galaxy 15.10 instance was locally installed on a server machine. WolfPsort, TMHMM, and SignalP were obtained from CBS prediction servers (https://services.healthtech.dtu.dk/, accessed Apr. 15, 2020). NLStradamus and PredictNLS were used in parallel to determine nuclear localization signals. Each dataset from RNA-seq, corresponding to a different population, was then processed through a pipeline designed to select sequences containing a signal peptide, no transmembrane segment, no nuclear localization signal, and structural features consistent with active secretion via classical or non-classical secretory pathways.

### qPCR Analysis

RNA was extracted from cardiac cells isolated from MI and SHAM C57BL/6J mice after 7 days from surgery using the RNAqueous Micro Kit (AM1931, Invitrogen), as per the manufacturer's instructions. Then, 500 ng of extracted RNA was subjected to reverse transcription using the SuperScript IV VILO kit (11756050, Invitrogen) as per the manufacturer's instructions. The resulting cDNA was subjected to qPCR using SYBR Select Master Mix (4472908, Applied Biosystems) on Quant Studio 3 Real-Time PCR system (Thermo Fisher) as per the following condition: 95° C. for 10 min, 40 cycles of 95° C. for 15 s and 60° C. for 1 min, followed by 95° C. for 10 s and 60° C. for 1 min. The expression of target gene was analyzed using the −2^{ΔΔCT} method following normalization to RPL13 expression.

### HEK293 Cell Culture and Transfection

HEK293 cells were cultured at 37° C. in the presence of 5% CO₂ in DMEM GlutaMax™-1 (Life Technologies) supplemented into 10% FBS and 1% penicillin and streptomycin. HEK293 cells were plated at 6×10⁵ cells/well in 6 well-plates in a medium without antibiotics. After 24 h, transfection of expression plasmids (Origene and Genscript) was performed with Lipofectamine® 2000 (Life Technologies) according to the manufacturer's protocol using 2 μg of plasmids and 6 μL of Lipofectamine® 2000 diluted in Opti-MEM (Life Technologies). The cells were cultured for 2 days and then serum starved for 8 h prior to the collection of conditioned media and centrifugation at 200×g for 10 min. Supernatants were stored at −80° C. until MEF treatment.

### Isolation of Mouse Embryonic Fibroblasts

Primary MEFs were isolated from 13.5-days post coitus C57bL/6J mouse embryos. The pregnant females were euthanized by cervical dislocation and embryos were surgically excised and separated from maternal tissues and the yolk sac in ice-cold phosphate-buffered saline (PBS). Embryos were then decapitated and eviscerated (removal of the heart, spleen, liver and intestine). The bodies were washed in ice-cold PBS to remove blood before being finely minced in a Petri dish without PBS. Samples were incubated for 15 min at 37° C. in the digestion solution (0.05% trypsin-EDTA solution [Life Technologies], 0.1 mg/mL DNase 1 [Sigma]). The suspension was allowed to settle. The supernatant was drawn off, mixed with MEF culture medium (DMEM 4.5 g/L D-glucose [Life Technologies], 10% FBS, 1% penicillin-streptomycin, 1% non-essential amino acid [Life Technologies]) and centrifuged for 5 min at 200×g. After centrifugation, the pellet containing MEFs was resuspended in MEF culture medium. The pellet from the tissue digestion was resuspended in the digestion solution and incubated for 15 min at 37° C. The cells were allowed to settle, the supernatant was drawn off and processed as previously described. The cells from the first and second digestion steps were pooled and then plated in Petri dishes. Each Petri dish received a volume of cell suspension equivalent to 1.5 embryos.

After 12 h, the culture medium was changed to remove non-adherent cells and debris. The MEFs were passaged upon reaching 80% confluence. MEFs were harvested by trypsinization, centrifuged, and resuspended in a freezing medium (DMEM 4.5 g/L D-glucose, 1% penicillin-streptomycin, 10% dimethyl sulfoxide). Primary MEFs were cultivated between passage 0 and 4.

### Western Blot Analysis

Proteins were extracted from frozen mouse heart tissues using a Dounce-Potter homogenizer into ice-cold radioimmunoprecipitation assay (RIPA) buffer (50 mM Tris pH 7.4, 150 mM sodium chloride, 1% IGEPAL CA-630, 50 mM deoxycholate, and 0.1% sodium dodecyl sulfate [SDS]) supplemented with 1% anti-proteases (Sigma-Aldrich), 1% anti-phosphatase inhibitors (Phosphatase Inhibitor Cocktail 2 and 3, Sigma-Aldrich), and 1 mM sodium orthovanadate. After 1 h incubation at 4° C., the homogenate was centrifuged for 15 min at 15,300×g and 4° C. and the supernatant containing proteins was collected. Cardiac PW1⁺ cells from 22 mice and PW1⁻ cells from 16 mice were pooled, centrifuged at 500×g for 15 min at 4° C., and lysed in urea-thiourea buffer (5 M urea, 2 M thiourea, 50 mM dithiothreitol [DTT], and 0.1% SDS in PBS, pH 7.4). Proteins were extracted as described above. Protein concentrations for all samples were determined using a Bradford-based protein assay (Bio-Rad).

After isolation of cardiomyocytes and non-cardiomyocytes from the adult mouse hearts, as previously described⁷, the proteins were denatured for 10 min at 70° C. before loading on a NuPAGE™ Novex® 4-12% Bis-Tris gel (Life Technologies). After 3 h electrophoresis, proteins were transferred onto nitrocellulose membranes using the Trans-Blot Turbo Transfer System (Bio-Rad) and stained with 0.1% Ponceau S (w/v in 5% acetic acid) to assess transfer quality and homogeneous loading. Membranes were blocked for 1 h in Tris-buffered saline with 0.1% Tween-20 (TB S-Tween) containing 5% skim milk with constant shaking and then incubated for overnight at 4° C. with primary antibodies specific for GDF3 (1:1000 for tissue and 1:500 for plasma, Abcam) and FLAG (1:1000, Sigma-Aldrich) diluted in 5% skim milk/TB S-Tween. After washing, the membranes were incubated for 1 h at room temperature (23° C.) with horseradish peroxidase (HRP)-conjugated secondary antibodies diluted in 5% skim milk/TB S-Tween. Membranes were then washed and incubated for 5 min with SuperSignal™ West Pico PLUS Chemiluminescent Substrate (Life Technologies) before imaging with the Chemidoc® XRS+ camera (Bio-Rad) and analysis using the Image Lab™ software.

### ELISA

GDF3 levels were measured by GDF3 sandwich ELISA assay (GenWay, GWB-KBBHW6) following the manufacturer's instructions. Briefly, standards and diluted samples (1:16 in standard diluent) were added to an anti-GDF3 microplate (pre-coated plate with an antibody specific for GDF3) and incubated for 1 h at 37° C. After removing standards and samples, a biotinylated GDF3 detector antibody was applied. The plate was incubated for 1 h at 37° C. Wells were washed and then incubated at 37° C. with an avidin-HRP conjugate for 30 min. Finally, after extensive washing, the wells were incubated with the 3,5,3′,5′-tetramethylbenzidine (TMB) substrate for 15 min in the dark at 37° C. The blue color product from the oxidation of TMB substrate changed into yellow after reaction termination with the addition of stop solution and incubation at 37° C. for 15 min. Absorbance at 450 nm was quantitatively proportional to the amount of GDF3 captured in well and measured using microplate reader.

### PREGICA Cardiac MRI Sub-Study

We used banked plasma from 80 patients with a first STEMI and who were enrolled in the prospective PREGICA cardiac MRI sub-study (Predisposition Genetical in Cardiac Insufficiency, clinicaltrials.gov identifier NCT01113268. Details of the study have been described previously (Garcia R, Bouleti C, Sirol M et al. VEGF-A plasma levels are associated with microvascular obstruction in patients with ST-segment elevation myocardial infarction. Int J Cardiol 2019; 291:19-24). Briefly, the study involved 6 cardiology centers in France and enrolled patients between 18 and 80 years old referred for a first STEMI between 2010 and 2017 and seen within the first 24 h after symptoms onset. STEMI was defined by the presence of ST-segment elevation on the ECG, significant rise of troponin (≥3 fold higher than the upper limit reference) and the presence of at least 3 akinetic LV segments on the initial trans-thoracic echocardiography. Patients were not included if they had permanent atrial fibrillation, a diagnosis of previous MI or a history of cardiac disease. All patients had coronary angiography and primary PCI in the first 24 h. Cardiac MRI was performed using a 1.5-T unit at day 4±2 after hospital admission and at 6-month follow-up in a subset of patients defining the CMR substudy of the PREGICA cohort. A standardized MRI protocol was followed in all centers and images were centrally analyzed. Cine images were acquired using a breath-holf steady-state free-precession sequence in long-axis and short-axis views. A stack of short-axis slices covering from the atrioventricular ring to the apex was used to derive left ventricular (LV) volumes, and ejection fraction (EF). Ten minutes after intravenous injection of gadolinium-based contrast agent, late gadolinium enhancement (LGE) images were acquired using a breath-hold segmented T1-weighted inversion-recovery gradient-echo sequence in the same long-axis and short-axis views of cine images. LGE images were assessed for infarct size. Blood samples were drawn at the same time than cardiac MRI. GDF3 was quantified on available plasma drawn at day 4 (n=80) using ELISA assays. The study was approved by the institutional rewiew board, and all patients provided written informed consent.

### Statistical Analysis

Mouse and in vitro studies. The number of samples (n) used in each experiment is recorded in the text and figure legends. All experiments were performed independently at least twice. The data are expressed as mean±standard deviation (SD). Quantitative data were analyzed using one-way analysis of variance (ANOVA, Kruskal-Wallis test) and pair-wise comparisons with Dunnett's post-hoc test for multiple comparisons. The Mann-Whitney U test was used for comparing continuous variables between two groups.

Analyses of PREGICA cardiac MRI sub-study. P-value were obtained from Chi-Square test statistics for binary variables and by using the Mann-Whitney U test for comparing continuous variables between two groups. The association between GDF3 levels and the likelihood of presenting adverse cardiac remodeling was assessed by linear regression models, with additional adjustment for age and sex.

All statistical analyses were performed using GraphPad Prism 8.0 (GraphPad Software, Inc., San Diego, CA). A value of P<0.05 indicated statistical significance.

### Results

PW1⁺ cells from ischemic hearts release factors that promote fibroblast proliferation. We subjected adult PW1^{nLacZ} reporter mice to ischemic cardiac injury by left anterior descending coronary artery (LAD) ligation, as previously described^{6,7}. Hearts were harvested from mice with MI as well as SHAM-operated mice at day 7 post injury, and PW1⁺ cells were isolated by fluorescence-activated cell sorting (FACS) (not shown). Following cultivation for 5 days, the conditioned media from these cells were collected and used to culture mouse embryonic fibroblasts (Miffs) for 24 h (not shown). The effect of the conditioned media on the proliferation of MEFs was evaluated using the CyQUANT™ Cell Proliferation Assay. The result of cell proliferation assay revealed the significant increase in the proliferation of MEFs incubated with the conditioned media from PW1⁺ cells isolated from ischemic hearts as compared with those treated with the conditioned media from control cells or PW1⁺ cells from SHAM-operated hearts (not shown). There was no significant increase in response to conditioned media from PW1⁻ cells (not shown). These observations suggest that the activated PW1⁺ cells from the ischemic heart release pro-proliferative factors, which may induce the proliferation of resident fibroblasts.

### RNA-Sequencing (RNA-Seq) and Bioinformatic Analyses Predict Candidate Biomarkers Involved in the Paracrine Action

The transcriptome of FACS-isolated PW1⁺ cells from SHAM and MI mice was characterized by RNA-seq to investigate the influence of the ischemic heart environment on the paracrine potential of PW1⁺ cells. We filtered, aligned, and quality-controlled RNA-seq output files to obtain a list of transcripts showing the greatest signal intensities (not shown). We performed a comparative analysis to understand the disease-induced alterations in the secretory behavior of cardiac PW1⁺ cells and shortlisted candidates with more than two-fold higher expression in ischemic conditions than in normal condition (not shown). We then examined the predicted amino acid sequences of the corresponding genes by a series of bioinformatic algorithms to identify secretory proteins. We considered proteins with a predicted N-terminal endoplasmic reticulum (ER)-targeting signal peptide but without predicted transmembrane domains and intracellular localization signals (i.e., no ER retention signal, mitochondrial targeting peptide, or nuclear export signal) (not shown). Progressive filtering rendered a total of 24 secreted proteins overexpressed by cardiac PW1⁺ cells under ischemic conditions (not shown). We next confirmed the significant increase in the expression of 12 of these 24 candidates in the ischemic heart (remote or infarct zones) as compared with normal heart by quantitative polymerase chain reaction (qPCR) (not shown).

In comparison with the secretome of control cells, the secretome of MI-activated PW1⁺ cells comprised several growth factors, cytokines, and enzymes as well as some poorly characterized factors (not shown). The secretion of the growth factor GDF3, cytokines such as NDP and CCL8, and enzymes such as CELA1 and PRTN3, were more than two-fold higher in MI hearts than in SHAM hearts (not shown).

### Transfection Experiment Confirms the Candidates with Proliferative Effects on Fibroblasts

We then investigated the effects of candidates on the proliferation of cultured embryonic fibroblasts and adult cardiac fibroblasts. We selected six candidates (CCL8, CELA1, GDF3, NDP, PRNT3, PROK2) that were possibly associated with cell proliferation, as evident from their Gene Ontology biological functions. Reciprocally, we excluded the lipoproteins APOC2, APOC4, and SAA3, the coagulation factor F10, and the poorly characterized C1QTNF3 and DMKN. We separately cloned the cDNAs of these six proteins into mammalian expression plasmids, which were then used to transfect HEK-293 cells (not shown). FLAG epitope-tagged fibroblast growth factor 23 cDNA served as positive control, while empty vector was used as negative control. After 48 h from transfection, the conditioned media were collected, tested to confirm the overexpression of the secreted proteins (not shown), and then used to incubate serum-starved MEFs. Evaluation of cell proliferation rate after 24 h treatment revealed four factors, namely, growth differentiation factor-3 (GDF3), norrin cystine knot growth factor (NDP), prokineticin 2 (PROK2), and chymotrypsin-like elastase family member 1 (CELA1), that significantly induced the proliferation of MEFs as compared with control treatment (not shown). The proliferative effects of three (GDF3, NDP and PROK2) of the four candidates were further confirmed using freshly isolated adult cardiac fibroblasts (not shown). Thus, cell proliferation assays facilitated the selection of three candidates GDF3, NDP, and PROK2 from 12 overexpressed markers.

Of these three remaining candidates, GDF3 (also known as Vg-related gene 2) combined the largest over-expression in the ischemic hearts and one of the most important increase of fibroblast proliferation. GDF3 is a member of the TGF-(3 superfamily that is composed of 366 amino acid residues. Human and mouse GDF3 show 76.6% nucleotide homology and 69.3% peptide identity¹². The predicted amino acid sequence comprises a signal sequence for secretion at the hydrophobic NH₂-terminus, a prodomain that facilitates cysteine-mediated disulfide bond formation with another family member, and a putative proteolytic processing site at 114 amino acid residue (not shown). The cleavage of GDF3 at this residue generates a mature GDF3 protein, which is 114 residue long¹¹. GDF3 plays an important role during early development in mice and humans¹³, but its expression is low in adult organs and particularly negligible in the adult heart^{10,14,15}. While the functions and implications of GDF3 in the adult heart are yet unknown, GO biological functions suggest its involvement with the SMAD protein signal transduction pathway that is very relevant to the process of cardiac fibrosis. This is consistent with the highest proliferative effect of GDF3 among all candidates on MEFs (not shown). Our transfection experiment (not shown) confirms that GDF3 is a secreted protein, as indicated by the band of full-length protein on the western blot of supernatants (not shown). Together these observations suggest the potential role of GDF3 in the regulation of fibroblast proliferation in the scarring tissue and encouraged us to investigate the expression profile of GDF3 in MI hearts of mice and humans.

### GDF3 Level Increases in the Plasma and Infarcted Area of the Mouse Heart After MI

Based on the transcriptome results, we sought to evaluate the expression of GDF3 and determine its cellular sources in the whole hearts by western blotting. We detected the mature form of GDF3 in both neonatal and adult normal hearts (not shown). In particular, GFD3 was expressed only in the non-cardiomyocyte fraction and not by cardiomyocytes in the adult heart (not shown). Further analysis of the non-cardiomyocyte fraction confirmed the specific expression of GDF3 in PW1⁺ cells but not in the PW1⁻ cell population of normal hearts (not shown). To investigate any dysregulation in the expression of GDF3 in the mouse heart following MI, we generated a permanent LAD mouse model and excised hearts after 7 days. We separately analyzed GDF3 expression in the infarcted area corresponding to the scar tissue and the remote area. Western blotting confirmed the higher expression of GDF3 in the infarcted area of MI hearts than in the corresponding areas of SHAM hearts (not shown). Consistent with our previous observations and with the fibrogenic fate of cardiac PW1⁺ cells in response to MI, this result indicates that GDF3 is produced at the site of infarction and suggests an involvement in the scarring process following MI.

### GDF3 is a Circulating Factor Secreted Post-MI

Considering the secretory nature of this protein, we investigated if free GDF3 could be detected in the circulation by analyzing plasma samples from MI and SHAM mice. Western blot analysis confirmed the presence as well as higher level of mature GDF3 in the plasma of MI mice than in the plasma of SHAM mice (not shown). We performed an enzyme-linked immunosorbent assay (ELISA) specific for GDF3 to investigate kinetic changes over time in the circulating levels of GDF3 in the mouse plasma by ELISA. The secreted protein level increased from day 0 to day 2 and decreased thereafter until day 7 post-MI.

Overall and in line with the fibrogenic fate of cardiac PW1⁺ cells, these results indicate that GDF3 may be a novel cardiokine secreted by these cells that may be related to adverse cardiac remodeling post-MI.

### Circulating GDF3 Level Serves as a Marker of Post-MI Adverse Remodeling in Humans

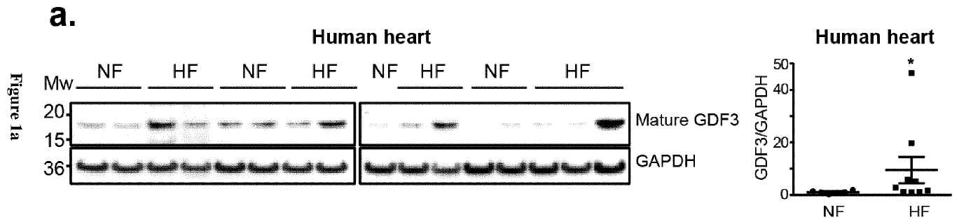

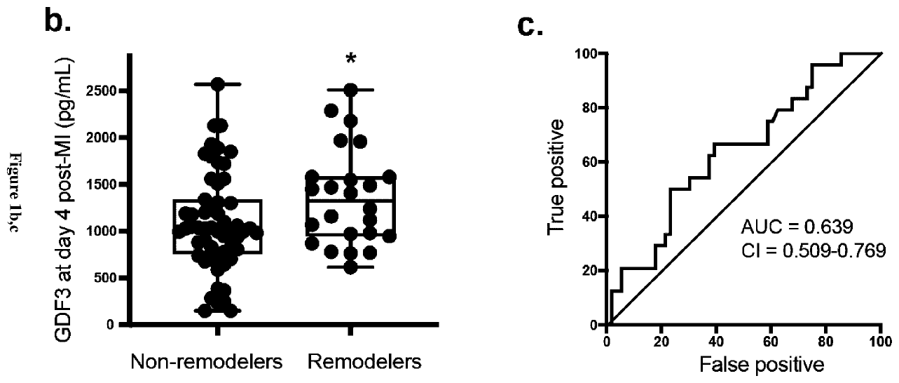

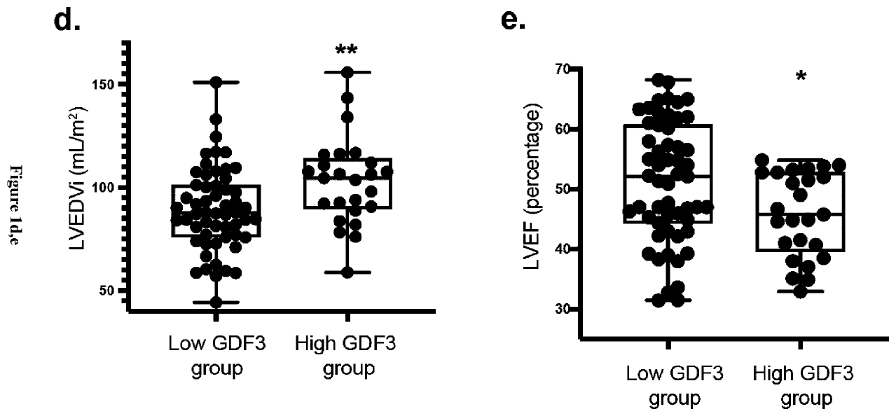

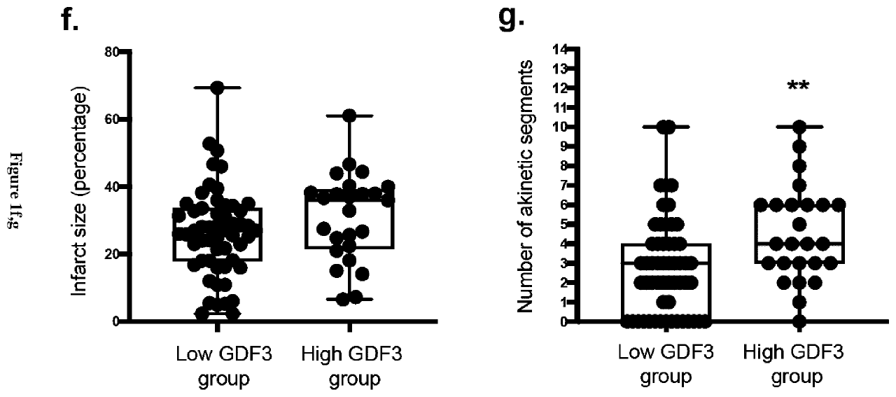

To investigate the clinical relevance of our findings in the murine MI model, we first assessed GDF3 expression on left ventricular cardiac tissue samples from failing ischemic hearts and non-failing hearts of patients. Western blot analysis revealed the stronger expression of GDF3 in the failing hearts than in the non-failing hearts (FIG. 1a), indicating the upregulation in the expression levels of GDF3 in the heart is a conserved response to MI.

We then asked whether elevated circulating GDF3 levels can be linked to adverse cardiac remodeling post-MI. We analyzed circulating GDF3 levels in 80 patients with a first acute ST-elevation myocardial infarction (STEMI), seen <24 h after symptom onset and treated by primary percutaneous coronary intervention (PCI) (Predisposition Genetical in Cardiac Insufficiency [PREGICA] patient collection, NCT01113268). Patients had an initial clinical and biological evaluation at day 4 and serial cardiac magnetic resonance imaging (MRI) at 4 days and at 6 months after angioplasty. The details of the inclusion/exclusion criteria are mentioned at https://clinicaltrials.gov/ct2/show/NCT01113268. The baseline characteristics of these patients are shown in Table 1.

We first defined adverse cardiac remodeling as a >20% increase in left ventricular end-diastolic volume (LVEDV) indexed for body surface area (LVEDVi, ml/m²) at 6 months as compared to the initial evaluation on cMRI. Patients were accordingly classified as remodelers (n=24) and non-remodelers (n=56). GDF3 measured at day 4 post-PCI was detectable in the plasma of these patients and levels were significantly higher in remodelers than in non-remodelers (1364±521 versus 1090±532 pg/mL, p=0.033) (FIG. 1b). After adjusting for age and sex, one standard deviation (SD) increase of GDF3 levels was associated with an increased risk of adverse remodeling (Odds Ratio (OR)=1.76 [1.03-3.00], p=0.037). Plasma GDF3 levels did not demonstrate any statistical difference (p>0.10) according to sex, smoking, personal history of hypertension and diabetes. Of note, GDF3 levels were moderately correlated to CRP (p=0.13) and Hb1AC (p=0.18), even if these correlations did not reach statistical difference (p=0.28 and p=0.12, respectively). To better assess the relation between cardiac remodeling and plasma GDF3 levels, we first divided patients into 4 quartiles of GDF3 levels (measured at day 4 post-MI) and compared LVEDVi and LVEF measured on cardiac MRI 6 months after MI between the quartiles. We found that patients with the highest GDF3 levels (i.e. quartile 4) had significantly higher LVEDVi and lower LVEF as compared to patients with lower GDFR3 levels. We next performed a receiver operating characteristic (ROC) curve analysis to determine if GDF3 level could help distinguishing the two groups. The area under the ROC curve of the age and sex adjusted model was significant (0.69 [0.56-0.82] (p=0.05)) and yielded a cut-off value of 1375 pg/mL at a likelihood ratio of 2.154 and sensitivity and specificity of 50% and 77%, respectively (FIG. 1c).

We then classified patients according to this cut-off value (≥1375 pg/mL called high GDF3, n=25 and <1375 pg/mL called low GDF3, n=55). Table 2 reports the main characteristics and cardiac MRI findings at baseline and 6 months after MI in both groups. There was no significant imbalance in the main cardiovascular risk factors between the two groups. The delay between symptom onset and coronary disocclusion was significantly longer (by 1.2 h) in patients with high GDF3 levels (P<0.05). However, the peak of troponin, a surrogate marker of myocardial necrosis, was significantly lower in the high GDF3 group. In terms of cardiac remodeling, patients with high GDF3 showed a non-significant trend of higher cardiac dilation at day 4 post-MI than those with low GDF3. The values of LVEDVi were however significantly higher (P<0.005) and pathological (normal value<82 mL/m2) at 6 months post-MI in patients with high GDF3 (FIG. 1d), indicative of adverse cardiac remodeling along with the development of cardiac dilation. These patients also showed a significant decrease (p<0.05) in LVEF at day 4 and 6 months (FIG. 1e), suggesting the reduced recovery of contractile functions post-MI in these patients. While total infarct size (percentage total cardiac mass) was not significantly different between the two groups (FIG. 1f), patients with high GDF3 levels had a higher proportion of akinetic segments on cardiac MRI at 6 months than those with low GDF3 levels (FIG. 1g). This result indicates the more important pathological transformation of infarcted areas into non-contractile scars in patients with high GDF3 levels, emphasizing its diagnostic importance as a marker of adverse cardiac remodeling after MI.

### Discussion

Acute myocardial infarction characterized by left ventricular remodeling may progress toward development of heart failure¹⁶. Markers reflective of myocardial damage may not predict long-term left ventricular remodeling (troponin and creatine kinase) or suffer from insufficient clinical data (galactin-3 and soluble interleukin-1 receptor-like 1)^{17,18}. For instance, galectin-3 was shown to be involved in fibrosis and inflammation and independently associated with incident peripheral artery disease in an observational study with Whites and Blacks only and without eliminating the effects of confounding factors. Thus, it is imperative to discover potential makers informative of preclinical HF to identify patients with an increased risk of HF and for timely disease management.

In an attempt to contribute to wound healing, the cardiac ECM undergoes constant remodeling upon injury¹⁹. Interestingly, the concept of ECM regulation through key molecules involved in intercellular communications has only recently emerged. Here, we focused on cardiac PW1⁺ cells, a cellular subpopulation that is suspected to orchestrate the reparative process in tissues, including the heart^{6,7}. We investigated major differences in the secretome of these PW1⁺ stromal cells in ischemic mouse hearts. Of note, the pro-proliferative effect observed with conditioned media from cardiac PW1⁺ cells isolated from ischemic mouse hearts was not observed with cardiac PW1⁻ cells nor with cardiac PW1⁺ cells isolated from normal mouse hearts. RNA-sequencing and bioinformatic analyses confirmed the upregulation in the expression of several factors in MI hearts, (12 secreted factors by MI-activated cardiac PW1⁺ cells), particularly GDF3, PROK2, NDP, which were confirmed to exhibit about seven-fold, three-fold, and three-fold expression upregulation following MI in qPCR validation experiment (not shown). Moreover, the 12 dysregulated candidate markers confirmed by qPCR validation are mostly enriched in GO biological processes such as angiogenesis, inflammation, chemotaxis, and proliferation, thus confirming the crucial response of PW1⁺ cell population to MI.

MI is characterized with an acute inflammatory response involved in myocardial repair²⁰; however, uncontrolled chronic inflammation causes excessive damage and fibrosis, eventually leading to the loss of cardiac function²¹. Cardiac inflammation and endothelial dysregulation are related to the remodeling of the extracellular matrix (ECM)²², and TGF-β pathways have been consistently highlighted as the key molecular mediators of cardiac fibrosis^{23,24}.

As a member of TGF-β superfamily, GDF3 was initially shown to participate in early embryonic development, muscular development, adipose tissue homeostasis, and energy balance through its interaction with activin receptor-like kinase type I receptor B (ACVR1B, ALK4) and ACVR1C (ALK7) receptors²⁵. Recent studies have highlighted the critical role of GDF3 in macrophage function and inflammation cascade. Wang et al. recently described the role of GDF3 in macrophage polarization and endotoxin/sepsis-induced cardiac injury²⁶. In the present study, we identified the previously unrecognized function of GDF3 in cardiac fibrosis and demonstrate the dynamic changes in GDF3 levels in the blood and hearts of mice and humans following MI.

This is the first report to investigate the prognostic potential of GDF3 in a cohort of post-MI patients. Interest in GDF3 as a marker of adverse cardiac remodeling arose from our pre-clinical study in a murine model of MI. We observed a seven-fold increase in the mRNA expression of GDF3 and about a two-fold increase in the circulating level of GDF3 in the mouse heart within 7 days from MI. These results were replicated in clinical samples derived from patients with MI. Thus, our results confirm the transient increase in GDF3 levels in murine MI model and highlight the novel pivotal role of this marker as a paracrine factor secreted by cardiac PW1⁺ cells in the process of regulation of the properties of the scar tissue and cardiac fibroblasts following MI. Therefore, circulating GDF3 level may be considered while gauging the risk of adverse outcome in patients after MI.

In our previous report, we showed reduction in TGF-β activation in vitro and cardiac fibrosis post-MI in vivo following pharmacological blockade of αV-integrin on activated cardiac PW1+ cells⁷. This observation and the involvement of GDF3 in TGF-β signaling¹¹ prompt the contribution of GDF3 to adverse cardiac remodeling post-MI. We speculate the participation of GDF3 in the inflammatory cascade during/post MI and support the concept of early intervention of GDF3 functions in the inflammatory cascade to prevent the myocardial damage. Risk stratification at an early stage after MI is challenging yet useful to tailor personalized treatment regimen in the future. Thus, our study paves a strong foundation for future studies directed to target GDF3 in the treatment of MI, supported by the association we found between circulating GDF3 levels, post-MI scarring processes, and cardiac functions.

Our clinical study is limited by the small sample size, as we specifically focused on post-MI patients with cardiac MRI evaluation for cardiac remodeling, an investigation that is not routinely performed in these patients. Our results indicate that patients with high GDF3 levels develop adverse cardiac remodeling based on imaging surrogates, but further studies would be required to validate its prediction on heart failure and cardiovascular outcomes. However, a large proportion of patients with high GDF3 levels had a significantly decreased LVEF (<50%) 6 months after MI. Further, while we only focused on the potential implications of GDF3, the role of other upregulated markers, particularly PROK2 and NDP, was not investigated and warrant future studies. Notably, we cannot exclude a potential synergy between these other secreted factors. Lastly, this study investigated the role of secreted factors on fibroblast proliferation as a primary mechanism underlying cardiac fibrosis. However, other mechanisms support the fibrotic transformation of the ischemic heart such as myofibroblast transformation²⁷ and immune-inflammatory response²⁸. The impact of GDF3 on these mechanisms merits further investigations.

### Conclusion

We show here that the upregulation of GDF3, a secreted protein, is detected in the plasma of mice and humans following MI. We interpret that the levels of circulating GDF3 correlates with the local cardiac production in response to MI and that higher GDF3 circulating levels would indicate higher proliferation of fibroblasts and higher fibrogenesis. Concordantly, we show that high levels of plasma GDF3 levels in a cohort of post-MI patients 4 days following MI corresponded with adverse outcomes measured 6 months later including cardiac dilation, limited recovery of contractile function and higher number of akinetic segments. These data suggest that higher circulating GDF3 levels can be used to identify patients who will develop adverse cardiac remodeling.

In conclusion, PW1⁺ cells from the ischemic heart release pro-proliferative factors, which induce proliferation of resident fibroblasts. One such factor, GDF3 may serve as a novel marker of adverse fibrotic remodeling in the heart tissue following MI. Its applicability in the clinical setting may allow for the identification of patients that have an increased risk of severe myocardial fibrosis and HF as well as better and more specific disease management.

### TABLES

TABLE 1
Baseline characteristics of patients

| Characteristics | Mean ± SD or N (%) | |
|---|---|---|
| Age (years) | 57.6 ± 10.3 | |
| Male (%) | 67 | (83%) |
| Body weight (kg) | 76.4 ± 13.5 | |
| Height (cm) | 171 ± 8 | |
| Body mass index (kg/m²) | 25.9 ± 4.1 | |
| Body surface area (body formula) | 1.92 ± 0.20 | |
| Pre-existing hypertension | 27 | (33.5%) |
| T2DM | 15 | (18.7%) |
| Dyslipidemia | 47 | (58.7%) |
| Delay between symptoms and PCI (hours) | 9.84 ± 3.36 | |
| LMCA as a culprit | 52 | (65%) |

T2DM, type 2 diabetes; LMCA, left main coronary artery; PCI, percutaneous coronary intervention; SD, standard deviation

TABLE 2
Main characteristics and cardiac RMI findings at
baseline and 6 months after MI in both groups

| | | | Patients | | |
|---|---|---|---|---|---|
| | Patients with | | with >1375 | | |
| Characteristics | GDF3 <1375 pg/mL | | pg/mL | | P value |
| Age (years) | 58.6 ± 8.6 | | 55.5 ± 13.2 | | 0.17 |
| Males, n (%) | 44 | (80%) | 23 | (92%) | 0.31 |
| Body weight (kg) | 74.5 ± 13.2 | | 79.9 ± 13.6 | | 0.08 |
| Height (cm) | 170 ± 7 | | 173 ± 8 | | 0.26 |
| BMI | 25.7 ± 4.2 | | 26.4 ± 3.8 | | 0.21 |
| Pre-existing | 21 | (38.9%) | 6 | (24%) | 0.06 |
| hypertension | | | | | |
| T2DM | 8 | (14.5%) | 7 | (28%) | 0.15 |
| Dyslipidemia | 36 | (65%) | 11 | (44%) | 0.07 |
| Active smokers | | | | | |
| Delay between | 9.6 ± 3.4 | | 10.8 ± 3.4 | | 0.049 |
| symptoms | | | | | |
| and PCI (hours) | | | | | |
| LMCA as a culprit | 35 | (63.6%) | 17 | (68.0%) | 0.13 |
| Biology at day 4 | | | | | |
| Troponin peak | 115 ± 117 | | 66 ± 90 | | 0.0052 |
| C-reactive | 26.8 ± 40.2 | | 35.9 ± 64.9 | | 0.55 |
| protein (CRP) | | | | | |
| Cardiac parameters | | | | | |
| (MRI) at day 4 | | | | | |
| LVEDVi (mL/m²) | 82.4 ± 16.6 | | 91.8 ± 16.1 | | 0.052 |
| LVESVi (mL/m²) | 43.9 ± 13.2 | | 52.4 ± 12.9 | | 0.0097 |
| LVEF (%) | 47.3 ± 8.4 | | 43.3 ± 7.4 | | 0.0296 |
| Number of akinetic | 4.66 ± 2.27 | | 5.64 ± 2.15 | | 0.035 |
| segments | | | | | |
| Cardiac parameters | | | | | |
| (MRI) at Month 6 | | | | | |
| LVEDVi (mL/m²) | 89.3 ± 20.2 | | 103.0 ± 21.3 | | 0.0065 |
| LVESVi (mL/m²) | 44.3 ± 17.1 | | 56.5 ± 17.1 | | 0.0032 |
| LVEF (%) | 51.3 ± 10.0 | | 45.8 ± 7.1 | | 0.0129 |
| Infarct size (%) | 26.3 ± 13.2% | | 31.2 ± 13.0 | | 0.08 |
| Number of akinetic | 2.80 ± 2.51 | | 4.52 ± 2.47 | | 0.0037 |
| segments | | | | | |
| Apical infarction | 32 | (58.1%) | 19 | (76%) | 0.12 |

| | | | |
|---|---|---|---|
| Cardiac parameters | | | |
| (Echo) at day 4 | | | |
| LVEDVi (mL/m²) | 51.3 ± 11.8 | 55.6 ± 9.0 | 0.11 |
| LVESVi (mL/m²) | 26.7 ± 7.8 | 31.0 ± 7.5 | 0.031 |
| LVEF (%) | 48.1 ± 7.6 | 44.4 ± 7.4 | 0.0223 |
| Cardiac parameters | | | |
| (Echo) at Month 6 | | | |
| LVEDVi (mL/m²) | 58.7 ± 13.9 | 67.4 ± 14.2 | 0.0188 |
| LVESVi (mL/m²) | 28.8 ± 10.8 | 34.5 ± 10.4 | 0.0205 |
| LVEF (%) | 52.2 ± 8.1 | 49.3 ± 8.7 | 0.14 |

T2DM, type 2 diabetes; LMCA, left main coronary artery; PCI, percutaneous coronary intervention; SD, standard deviation; BMI, body mass index; LVEDV, left ventricular end-diastolic volume; LVESV, left ventricular end-systolic volume; LVEF, left ventricular ejection fraction.

Statistical analysis was performed with Mann-Whitney non-parametric i-test for continuous variables and Chi-square (Fisher if n<5) for binary variables. P<0.05

### REFERENCES

Throughout this application, various references describe the state of the art to which this invention pertains. The disclosures of these references are hereby incorporated by reference into the present disclosure.
1. Frangogiannis, N. G. Regulation of the inflammatory response in cardiac repair. Circ Res 110, 159-173 (2012).
2. Gulati, A., et al. Association of fibrosis with mortality and sudden cardiac death in patients with nonischemic dilated cardiomyopathy. JAMA 309, 896-908 (2013).
3. Thygesen, K., et al. Third universal definition of myocardial infarction. Circulation 126, 2020-2035 (2012).
4. Scardovi, A. B., et al. Brain natriuretic peptide plasma level is a reliable indicator of advanced diastolic dysfunction in patients with chronic heart failure. Eur J Echocardiogr 8, 30-36 (2007).
5. Hinderer, S. & Schenke-Layland, K. Cardiac fibrosis—A short review of causes and therapeutic strategies. Adv Drug Deliv Rev 146, 77-82 (2019).
6. Yaniz-Galende, E., et al. Fibrogenic Potential of PW1/Peg3 Expressing Cardiac Stem Cells. J Am Coll Cardiol 70, 728-741 (2017).
7. Bouvet, M., et al. Anti-integrin alphav therapy improves cardiac fibrosis after myocardial infarction by blunting cardiac PW1(+) stromal cells. Sci Rep 10, 11404 (2020).
8. Yokota, T., et al. Type V Collagen in Scar Tissue Regulates the Size of Scar after Heart Injury. Cell (2020).
9. Li, Q., et al. The conditioned medium from a stable human GDF3-expressing CHO cell line, induces the differentiation of PC12 cells. Mol Cell Biochem 359, 115-123 (2012).
10. The hHuman Protein Atlas. https://www.proteinatlas.org/ENSG00000184344-GDF3/tissue. Accessed July 16.
11. McPherron, A. C. & Lee, S. J. GDF-3 and GDF-9: two new members of the transforming growth factor-beta superfamily containing a novel pattern of cysteines. J Biol Chem 268, 3444-3449 (1993).
12. Clark, A. T., et al. Human STELLAR, NANOG, and GDF3 genes are expressed in pluripotent cells and map to chromosome 12p13, a hotspot for teratocarcinoma. Stem Cells 22, 169-179 (2004).
13. Levine, A. J. & Brivanlou, A. H. GDF3, a BMP inhibitor, regulates cell fate in stem cells and early embryos. Development 133, 209-216 (2006).
14. Mouse Genome Informatics. http://www.informatics.jax.org/gxd/phenogrid/MGI:95686. Accessed, J., 2020.
15. BGEE. Gene Expression Data in Animals. https://bgee.org/?page=gene&gene_id=ENSG00000184344. Accessed July 17.
16. Sutton, M. G. & Sharpe, N. Left ventricular remodeling after myocardial infarction: pathophysiology and therapy. Circulation 101, 2981-2988 (2000).
17. Ponikowski, P., et al. 2016 ESC Guidelines for the diagnosis and treatment of acute and chronic heart failure: The Task Force for the diagnosis and treatment of acute and chronic heart failure of the European Society of Cardiology (ESC). Developed with the special contribution of the Heart Failure Association (HFA) of the ESC. Eur J Heart Fail 18, 891-975 (2016).
18. Tyminska, A., et al. Association of Galectin-3 and Soluble ST2, and Their Changes, with Echocardiographic Parameters and Development of Heart Failure after ST-Segment Elevation Myocardial Infarction. Dis Markers 2019, 9529053 (2019).
19. Schellings, M. W., et al. Absence of SPARC results in increased cardiac rupture and dysfunction after acute myocardial infarction. J Exp Med 206, 113-123 (2009).
20. Frangogiannis, N. G. The inflammatory response in myocardial injury, repair, and remodelling. Nat Rev Cardiol 11, 255-265 (2014).
21. Fontes, J. A., Rose, N. R. & Cihakova, D. The varying faces of IL-6: From cardiac protection to cardiac failure. Cytokine 74, 62-68 (2015).
22. Franssen, C., et al. Myocardial Microvascular Inflammatory Endothelial Activation in Heart Failure With Preserved Ejection Fraction. JACC Heart Fail 4, 312-324 (2016).
23. Khalil, H., et al. Fibroblast-specific TGF-beta-Smad2/3 signaling underlies cardiac fibrosis. J Clin Invest 127, 3770-3783 (2017).
24. Tschope, C. & Diez, J. Myocardial fibrosis as a matter of cell differentiation: opportunities for new antifibrotic strategies. Eur Heart J 40, 979-981 (2019).
25. Andersson, O., Korach-Andre, M., Reissmann, E., Ibáñez, C. F., & Bertolino, P. Growth/differentiation factor 3 signals through ALK7 and regulates accumulation of adipose tissue and diet-induced obesity. Proc Natl Acad Sci USA. 105, 7252-7256 (2008).
26. Wang L., et al. GDF3 Protects Mice against Sepsis-Induced Cardiac Dysfunction and Mortality by Suppression of Macrophage Pro-Inflammatory Phenotype. Cells 9, 120 (2020).
27. Weber, K. T., Sun, Y., Bhattacharya, S. K., Ahokas, R. A., Gerling, I. C. Myofibroblast-mediated mechanisms of pathological remodelling of the heart. Nat Rev Cardiol 10, 15-26 (2013).
28. Kino, T., Khan, M., Mohsin, S. The Regulatory Role of T Cell Responses in Cardiac Remodeling Following Myocardial Infarction. Int J Mol Sci. 21, 5013 (2020).
29. ANTI-INTEGRIN av THERAPY IMPROVES CARDIAC FIBROSIS AFTER MYOCARDIAL INFARCTION BY BLUNTING CARDIAC PW1+ STROMAL CELLS. Scientific Reports (2020).
30. Chen, C., Khaleel, S. S., Huang, H. & Wu, C. H. Software for pre-processing Illumina next-generation sequencing short read sequences. Source Code Biol Med 9, 8 (2014).
31. Li, B. & Dewey, C. N. RSEM: accurate transcript quantification from RNA-Seq data with or without a reference genome. BMC Bioinformatics 12, 323 (2011).
32. Yates, A., et al. Ensembl 2016. Nucleic Acids Res 44, D710-716 (2016).

1. A method of determining whether a patient who experienced a myocardial infarction has or is at risk of having adverse post-ischemic cardiac remodeling and treating the patient, comprising determining the level of GDF3 in a sample obtained from the patient and
administering a therapeutically effective amount of a GDF3 inhibitor to a subject identified as having high levels of GDF3.

2. The method of claim 1 wherein the sample is a blood sample, more particularly a serum sample.

3. The method of claim 1 wherein the level of GDF3 is determined 1, 2, 3, 4, 5, 6, 7, 8, 9, 10, 11, 12, 13, 14, or 15 days after the myocardial infarction.

4. The method of claim 1 that includes contacting the sample with an agent that selectively binds to the GDF3 protein.

5. The method of claim 4 wherein the agent is an antibody.

6. The method of claim 1 wherein the level of GDF3 is determined by an enzyme linked immunosorbent assay.

7. The method of claim 1 wherein high levels of GDF3 indicate that the probability that the patient has or is at risk of having adverse post-ischemic cardiac remodeling is high and conversely low levels of GDF3 indicate that the probability that the patient has or is at risk of having adverse post-ischemic cardiac remodeling is low.

8. The method of claim 1 that comprises the steps of i) quantifying the level of GDF3 in the sample obtained from the patient ii) comparing the level quantified at step i) with a predetermined reference value and iii) concluding that the patient has or is at risk of having adverse post-ischemic cardiac remodeling when the level quantified at step i) is higher than the predetermined reference value or inversely concluding that the patient does not have or is not at risk of having adverse post-ischemic cardiac remodeling when the content quantified at step i) is lower than the predetermined reference value.

9. A method of treating adverse post-ischemic cardiac remodeling in a patient who experienced a myocardial infarction comprising administering to the subject a therapeutically effective amount of a GDF3 inhibitor.

10. The method of claim 9 wherein the GDF3 inhibitor is an anti-GDF3 neutralizing antibody.

11. The method of claim 10 wherein the anti-GDF3 neutralizing antibody binds to the mature domain of GDF3.

12. The method of claim 11 wherein the anti-GDF3 neutralizing antibody binds to the amino acid sequence that ranges from the amino acid residue at position 251 to the amino acid residue at position 364 in SEQ ID NO:1.

13. The method of claim 4, wherein the agent selectively binds to a mature domain of GDF3 protein.


SEQUENCE LISTING (1 sequence)

SEQ ID NO: 1 - protein, 364 aa, Homo sapiens
MLRFLPDLAFSFLLILALGQAVQFQEYVFLQFLGLDKAPSPQKFQPVPYILKKIFQDREAAATTGVSRDLCYVKELGVRGNVLRFLPDQGFFLYPKKISQASSCLQKLLYFNLSAIKEREQLTLAQLGLDLGPNSYYNLGPELELALFLVQEPHVWGQTTPKPGKMFVLRSVPWPQGAVHFNLLDVAKDWNDNPRKNFGLFLEILVKEDRDSGVNFQPEDTCARLRCSLHASLLVVTLNPDQCHPSRKRRAAIPVPKLSCKNLCHRHQLFINFRDLGWHKWIIAPKGFMANYCHGECPFSLTISLNSSNYAFMQALMHAVDPEIPQAVCIPTKLSPISMLYQDNNDNVILRHYEDMVVDECGCG